(19) United States
(12) Patent Application Publication
(10) Pub. No.: US 2019/0274626 A1
(43) Pub. Date: Sep. 12, 2019

(54) HARNESS MOUNTED PATIENT MONITORING SYSTEM AND METHOD

(71) Applicant: Cloud DX, Inc., a corporation of Delaware, Brooklyn, NY (US)

(72) Inventors: Sara Ross-Howe, Campbellville (CA); Tarang Sheth, Oakville (CA); Lamiaa Amzil, Waterloo (CA); Josh Haid, Kitchener (CA); Cezar Morun, Kitchener (CA); David Widman, Oakville (CA); Robert Kaul, Brooklyn, NY (US)

(21) Appl. No.: 16/425,721

(22) Filed: May 29, 2019

Related U.S. Application Data
(63) Continuation-in-part of application No. 15/268,556, filed on Sep. 17, 2016, now abandoned.
Application No. 15/268,556 is a continuation-in-part of application No. 15/060,514, filed on Mar. 3, 2016, now Pat. No. 9,946,844.
Application No. 15/268,556 is a continuation-in-part of application No. 14/186,151, filed on Feb. 21, 2014, now Pat. No. 10,022,053.
Application No. 14/186,151 is a continuation-in-part of application No. 15/939,190, filed on Mar. 28, 2018.
(60) Provisional application No. 62/138,377, filed on Mar. 25, 2015.
(60) Provisional application No. 61/767,839, filed on Feb. 22, 2013.

(51) Int. Cl. A61B 5/00; A61B 5/0408; A61B 5/0205; A61B 5/1455; A61B 5/04; A61B 6/03

(52) U.S. Cl. CPC A61B 5/6822; A61B 5/04085; A61B 5/0205; A61B 5/14551; A61B 5/04012; A61B 5/02233; A61B 6/032; A61B 5/742; A61B 5/7405; A61B 5/486; A61B 5/7475; A61B 5/0006

(57) ABSTRACT

A patient monitoring device, system and method, based on a neck mounted monitoring harness configured to be capable of operating in a stand-alone mode. The neck-mounted harness is a rigid or semi-rigid U-shaped device, with its own independent processor, power source, and ECG circuitry, configured to be worn around the patient's neck with ECG electrodes, mounted on opposite ends of the U, configured to straddle opposite sides of the patient's sternum near the patient's heart. The device is also configured to interface with other devices such as patient ear worn oximeters and oscillometric blood pressure monitors. The device processor has the capability of independently operating the sensors, analyzing sensor data, and reporting results. The device is also configured to interface with various types of external computerized devices. The device can be configured to help to prepare patients for cardiac CT scans or other imaging scans, and this application is described in detail.

### CROSS REFERENCE TO RELATED APPLICATIONS

This application is a continuation in part of U.S. patent application Ser. No. 15/268,556, COMPREHENSIVE BODY VITAL SIGN MONITORING SYSTEM″, filed Sep. 17, 2016; application Ser. No. 15/268,556 was a continuation in part of U.S. patent application Ser. No. 15/060,514, “SYSTEMS AND METHODS FOR MONITORING PATIENT MEDICATION ADHERENCE”, filed Mar. 3, 2016, now U.S. Pat. No. 9,946,844; application Ser. No. 15/060,514 claimed the priority benefit of U.S. provisional application 62/138,377, “COMPREHENSIVE BODY VITAL SIGN MONITORING SYSTEM WITH NECK AND EAR MOUNTED DEVICE” filed Mar. 25, 2015; application Ser. No. 15/268,556 was also a continuation in part of U.S. patent application Ser. No. 14/186,151, “SIMULTANEOUS MULTI-PARAMETER PHYSIOLOGICAL MONITORING DEVICE WITH LOCAL AND REMOTE ANALYTICAL CAPABILITY”, filed Feb. 21, 2014, now U.S. Pat. No. 10,022,053; Ser. No. 14/186,151 claimed the priority benefit of U.S. provisional application 61/767,839, filed Feb. 22, 2013; this application is also a continuation in part of U.S. patent application Ser. No. 15/939,190, “AUGMENTED REALITY SYSTEMS FOR TIME CRITICAL BIOMEDICAL APPLICATIONS, filed Mar. 28, 2018; the entire contents of all of these applications are incorporated herein by reference.

### BACKGROUND OF THE INVENTION

### Field of the Invention

This invention is in the fields of medical instrumentation and medical diagnostic equipment and methods.

### Description of the Related Art

As the field of computerized tomography (CT) scanning has advanced, improved CT scanners have been developed that operate with ever increasing imaging speeds and spatial resolution. Due to these advances, in recent years, cardiologists and other medical professionals have found that cardiac CT scans are very useful for diagnosing coronary artery disease and other cardiac problems. However, although such improved CT scanners are now relatively fast, the human heart is even faster, and the motion of a normal heartbeat can still result in blurred cardiac images. With present CT scanners, in order to get sharp cardiac images, the patient's heartbeat rate (e.g. pulse) should preferably be less than about 65 beats per minute. Many patients have normal resting pulses significantly above this.

To get good cardiac CT images, in addition to a slow heartbeat rate, other patient physiological factors are also important. It is additionally important that the patient's heart is also beating regularly (e.g. be in sinus rhythm). This is because, for cardiac scans, the CT scanner is often configured to, during certain portions of the heartbeat, capture CT images of the heart over multiple heartbeats. If the patient's heart is not beating regularly, then again the quality of the cardiac CT scan image will suffer. Thus, before the cardiac CT scan begins, ECG data, such as at least single lead ECG data, is often needed to determine if the patient's heart is beating in a proper sinus rhythm (i.e. patient's heartbeat is regular). There are other considerations as well. The movement of the patient's chest during breathing can also result in blurred cardiac CT images. Thus to avoid image distortion due to movement in the chest area, during the cardiac CT scan, patients are often instructed to hold their breath for about 15 seconds. This breath holding can induce certain physiological alterations. When an individual holds their breath, their heartbeat rate will typically drop, and other physiological changes (e.g. potential changes in blood pressure, blood oxygen levels, regularity of the heartbeat) may occur. Some of these changes may be adverse to the patient's health, or adverse to the quality of the cardiac CT scan. Thus additional information, such as the impact of breath holding on the patient's heartbeat rate, ECG, and other parameters can be important for assessing if a patient is now properly prepared for the cardiac CT scan.

To lower the patient's heart beat rate to a low enough level to enable sharp cardiac CT scan images, without putting the patient at risk, present medical practice is to administer various drugs, such as the beta-blocker metoprolol, nitroglycerine, and/or other drugs. The goal is to safely and temporarily slow down the patient's heart beat rate during the scan. However, because of this drug use, still more continuous and accurate monitoring is required. Occasionally such drugs can lower the patient's blood pressure or blood oxygen levels too much, potentially compromising patient safety. As a result, information as to the patient's blood pressure or blood oxygen levels can also play an important role in determining which drugs to use, appropriate dose levels, appropriate times for administration, as well as determining if it is now more medically prudent to terminate further attempts at lowering the patient heartbeat rate.

As a result of these various advances, a new type of problem has emerged. CT scanners can operate relatively quickly, and if the patient is prepared properly, a cardiac CT scan can be performed over a time period that is only a fraction of an hour. By contrast, the current process of properly preparing a patient for cardiac CT scans can potentially take several hours. This process of preparing the patient also consumes a fair amount of healthcare professional time. For example, at present, two nurses, each working on the project full time, can process at best only about 15-20 patients for cardiac CT scans a day. By contrast, suitable cardiac CT scanning machines can easily scan more than 40 patients a day. Thus at present, patient preparation is a significant bottleneck in the cardiac CT scanning process. Due to this bottleneck, the cardiac CT machine can often sit idle, while patients in potentially urgent need of cardiac CT or other types of CT scans can't get access.

This lack of access is particularly problematic because cardiac CT scans can often detect cardiac problems that other diagnostic methods miss. Indeed, cardiac CT scanning is becoming increasingly recognized as being an important part of the standard of care for cardiac patients. However, due to this patient preparation bottleneck, the medical system is currently limited in its ability to adequately diagnose certain cardiac diseases, resulting in undesirable excess morbidity and mortality.

Patient Monitoring Devices

As technology has advanced, it has become increasingly feasible to monitor a plurality of different patient physiological parameters. Some of these developments are described by the applicant's earlier disclosures in U.S. patent application Ser. Nos. 61/767,839; 14/186,151; 62/138,377; 15/060,514; 15/268,556; 15/954,250 and 16/036,551, the entire contents of these patent applications are incorporated herein by reference.

### BRIEF SUMMARY OF THE INVENTION

The present invention was inspired, in part, by the insight that an improved version of the harness mounted user monitoring system, originally oriented more for home use, and previously described by the applicant in U.S. patent application Ser. Nos. 62/138,377; 15/060,514; 15/268,556; 15/954,250 might be useful in hospital and clinic situations as well. Indeed applicant's recent patent application 15/939,190 contemplated use of this harness mounted user monitoring system in conjunction with augmented reality systems for emergency rooms and other time-critical medical situations. Thus in some embodiments of the present invention, an improved version of the harness mounted user monitoring system previously taught by applicant's U.S. patent application Ser. Nos. 62/138,377, 15/060,514, and 15/268,556 and others is disclosed.

The present invention was also inspired, in part, by the insight that an improved version of the harness-based patient-wearable monitoring system previously described in applicant's U.S. patent application Ser. Nos. 62/138,377, 15/060,514, and 15/268,556 and 15/939,190 could also be useful in another important clinical application, specifically to help address the previously discussed problem of patient preparation for cardiac CT scans. Indeed, it appears that such an improved harness-based patient-wearable monitoring system may be useful in a wide variety of different clinical and home applications.

Thus in some embodiments, the invention may be an improved harness-based patient-wearable monitoring system (also occasionally termed an “ambulatory monitoring system”, a “monitor harness”, a “neck mounted monitor harness device”, and a “neck mounted device”) for a human user. This user may be a healthy home user without any diagnosed medical problems, but often may be a user with diagnosed medical problems as well. Thus, in this disclosure, the “user” is often referred to in the alternative as a “patient”. This system will typically comprise a neck mounted monitor harness comprising a harness processor, harness memory, battery, ECG circuitry, and a harness communications interface. This neck mounted monitor harness will typically comprise a rigid or semi-rigid U-shaped support (often comprising one or more silicone tubes that can bend enough by normal hand strength to facilitate mounting and dismounting of the harness from the neck of a human user, but otherwise in the absence of such hand force will maintain their shape during normal wearing by the user) with a center and two opposite ends.

These opposite ends are frequently referred to in the alternative as “pods”. In some embodiments, these pods may have their own computer display panels, connected to the harness processor, so that at least some signals or physiological parameters may be viewed directly without any need of external computerized devices. Thus in some embodiments, the neck mounted monitor harness can operate as a self-contained system. However in many embodiments, as will be described, the neck mounted monitor harness will interact with other devices as well.

Each opposite end or “pod” of this monitor harness will typically further comprise electrodes configured to make electrical contact with user's front chest skin on opposite sides of the user's sternum, thus providing at least a single lead ECG electrical connection enabling the ECG circuitry to implement an ECG sensor, such as a one-wire ECG sensor. Additional ECG electrodes or other type electrodes may also be provided by the monitor harness in alternate positions, such as in the portion of the harness that contacts the back of the user's neck.

The invention's monitor harness will typically further comprise any of a wired (e.g. a wired connector and wire) or wireless (e.g. Bluetooth, Wi-Fi) connection to at least one of a pulse oximeter and/or an oscillometric blood pressure monitor. This allows the harness processor to further control and receive data from additional sensors outside of the harness, such as the previously discussed pulse oximeter sensor and/or an oscillometric blood pressure sensor. In some embodiments, the invention's monitor harness may also be configured to connect directly, by hard wire or wirelessly, to the circuitry of the CT scanner, thus enabling the invention to co-ordinate CT imaging of the heart with the patient's heart beats.

In a preferred embodiment, the harness processor (often modern electronics employ multiple processors and/or multiple processor cores, so to simplify grammar, the term “harness processor” should be interpreted as reading on “at least one harness processor”) is configured to drive, over a plurality of patient heartbeats, the ECG sensor and any of the pulse oximeter sensor and/or oscillometric blood pressure sensor.

The monitor harness is also typically configured to use at least one harness communications interface (e.g. a connector and suitable hardware software drivers to a wired computer interface, or a wireless interface such as a Bluetooth or Wi-Fi transceiver and supporting drivers) to receive various system control data such as any of operating-analysis parameters, patient instructions, and physiological target data from an external computerized device. The harness processor will typically also be configured to store any of this system control data, such as the operating-analysis parameters, patient instructions, and physiological target data into the harness memory. The harness processor will then, for example, typically use these operating-analysis parameters to operate the ECG sensor(s) and other connected devices such as the pulse oximeter and/or the oscillometric blood pressure monitor. Other sensors, such as temperature sensors, motion sensors, audio sensors (microphones) can also form part of the monitor harness and can be controlled by the harness processor as well. These sensors can then collect various types of patent data.

In a preferred embodiment, the harness processor may be configured to do additional tasks, such as transmit any patient instructions to the user. This can be done by, for example, using the harness processor to transmit these patient instructions to an external computerized device, or display the patient instructions on the optional pod displays, or audio output by an optional built-in harness audio speaker, or audio output by, for example, an optional earphone that forms part of an ear-mounted pulse oximeter device.

The harness processor is often also further configured to do additional tasks such as to store data (e.g. sensor data) from any of the previously discussed sensors in the harness memory, and/or transmit this sensor data by using the harness communications interface and/or any optional built-in display screens, such as the optional pod display screens.

In a preferred embodiment, the harness processor is often further configured to use the previously discussed operating-analysis parameters to analyze data from some or all of the previously discussed sensors, and in some embodiments determine if the user's physiological parameters meet any previously established physiological criteria (for example, previously established criteria for being in a proper state for a cardiac CT scan). The harness processor may also be configured to use the harness communications interface to transmit at least some of this analyzed data to at least one external computerized device, such as a smartphone, tablet computer, hospital information system, and the like.

### BRIEF DESCRIPTION OF THE DRAWINGS

FIG. 1 shows an image of a patient wearing the invention's neck mounted monitor harness device (100) around her neck.

FIG. 2 shows a closer view of an embodiment of the invention's neck mounted monitor harness device (100).

FIG. 3 shows an alternate view of the invention's neck mounted monitor harness device.

FIG. 4 shows a more detailed 2D cross-sectional drawing of one of the pods (either 110 or 120) of the neck mounted monitor harness (100).

FIG. 5 shows a more detailed 3D drawing showing of one of the pods.

FIG. 6 shows an example touchscreen display from an external handheld computerized device.

FIG. 7 shows an example of a CT scanner, here a GE Lightspeed CT scanner.

FIG. 8 shows an inverted grayscale example of a cardiac CT scan.

FIG. 9 shows a simplified flow chart showing how the invention's neck mounted monitor harness device can be used to help prepare patients for cardiac CT scans.

FIG. 10 shows an electrical circuit diagram of one embodiment of the invention's neck mounted monitor harness device.

### DETAILED DESCRIPTION OF THE INVENTION

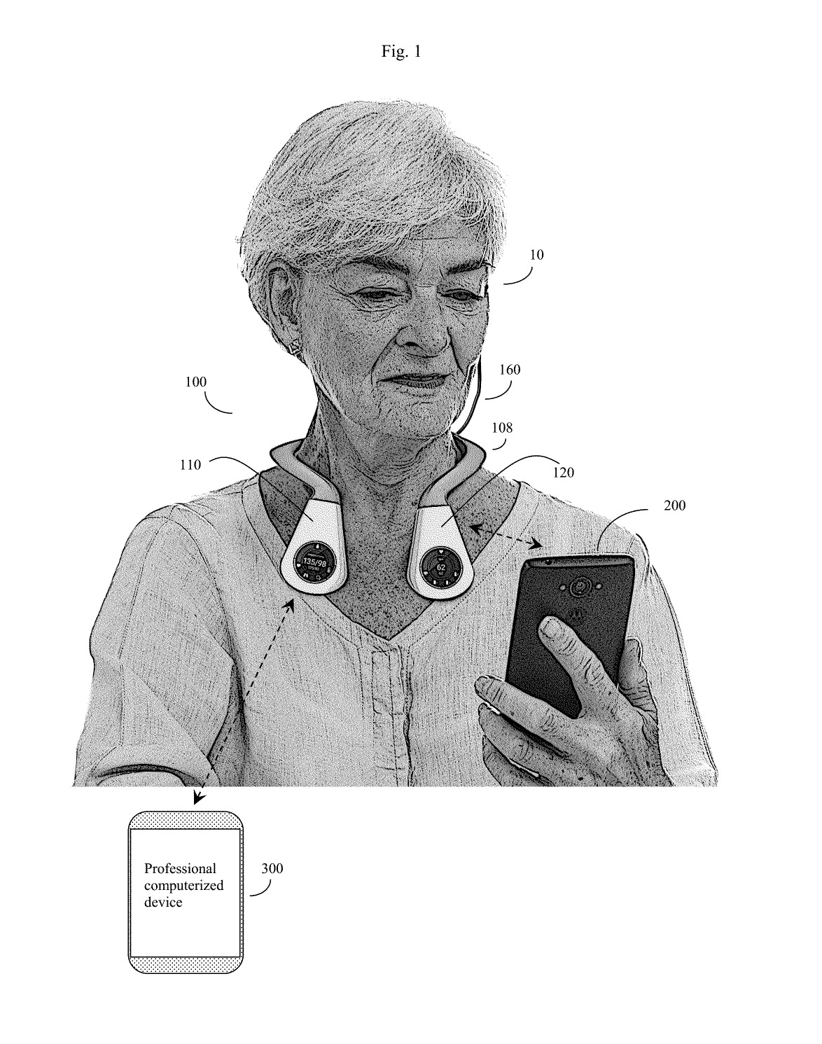

FIG. 1 shows an image of a user who is also a patient (10) wearing the invention's neck mounted monitor harness device (100) around her neck. The two ends of the harness each comprises a “pod” (110, 120) each “pod” comprising at least one ECG electrode, and the two pods are connected by a rigid or semi-rigid U-shaped support “harness” (108). This U-shaped support harness (108) can be made flexible enough to deform somewhat to enable the harness to be easily applied to or removed from the user's neck but is rigid enough so that it will otherwise maintain its shape while the patient is wearing it.

To help prevent the harness from falling off of the patient, it is helpful, but not essential, if the two opposite ends of the harness (110, 120) are separated by a gap that is narrower than the width of the patient's neck. At the same time, the harness needs to be easily worn by the patient, and also easily removed. Towards this objective, it is preferable to use an elastic semi-rigid harness that can elastically deform slightly in response to hand pressure to present a wider opening to slip around the user's neck, but then subsequently return to a configuration that is somewhat narrower than the user's.

Alternative harness configurations are also possible, however. Alternatively, with a somewhat wider opening, a rigid harness may also be used. A rigid, multiple-section, harness with easily attached and detached sections could also be used. A key consideration is that the harness should rigid enough so as not to deform significantly when the patient moves about while wearing the harness, or while the patient puts on or removes certain articles of clothing. Thus a harness consisting of merely a thin flexible conductive wire, for example, would not achieve these objectives.

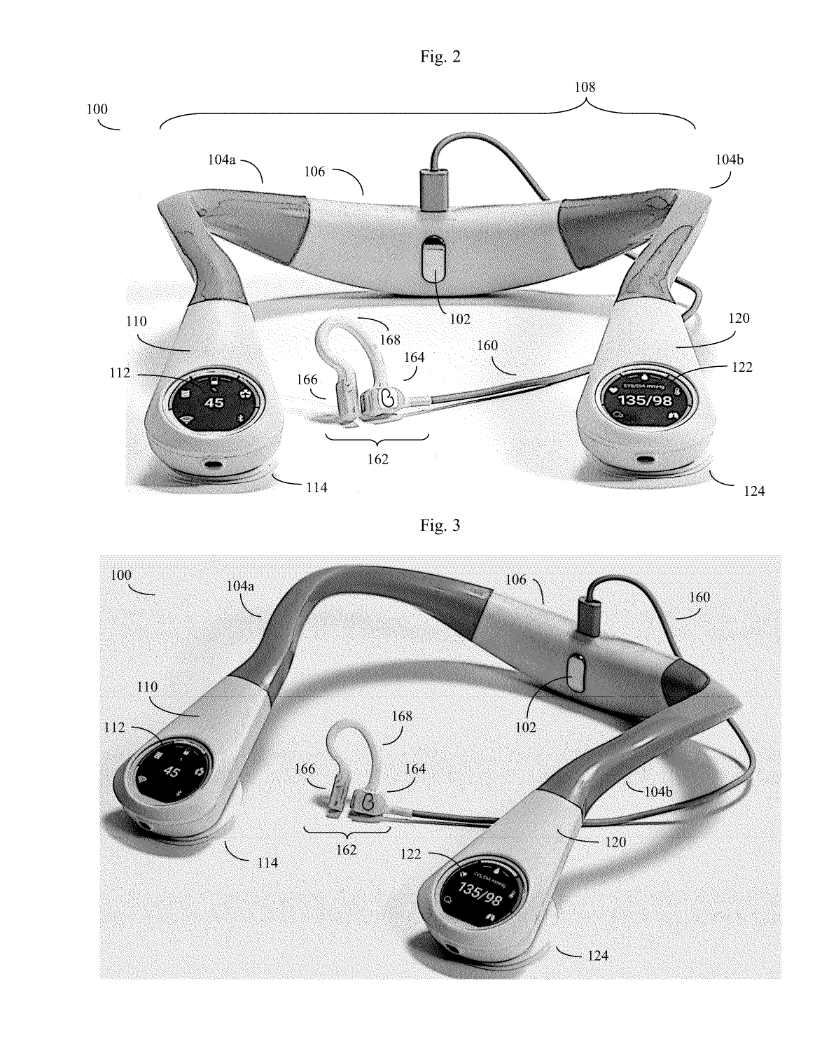

The harness device (100) is configured and positioned on the user (10) so that ECG electrode clips (and attached disposable wet or hydrogel ECG electrode pads, or dry electrodes), positioned underneath each of the two pods (110, 120) are positioned on opposite sides of the patient's sternum. This version of the neck mounted monitor harness device also comprises a wired connection (160) to an ear-mounted pulse oximeter (not shown in this figure, see FIG. 2, 162) that is configured to obtain pulse oximeter sensor data. In some embodiments, this ear mounted pulse oximeter may also comprise an audio output device (e.g. be configured as an audio earphone or earbud) so that the patient may more easily receive audio instructions from the harness device processor. The ear mounted pulse oximeter (162) may further comprise a temperature sensor or other sensors as desired.

The harness device (100) is further configured with a harness communications interface (here a wired interface, or a wireless transceiver such as a Bluetooth transceiver or Wi-Fi transceiver that can transmit ECG data and pulse oximeter data to an external computerized device). This nearby external computerized device may be a nearby patient-controlled smartphone (200). Alternatively this external computerized device may be a healthcare professional tablet computer (300), laptop or desktop computer, or even various types of computerized imaging scanners, wireless connection to an internet access point, hospital or clinic computer system, and the like.

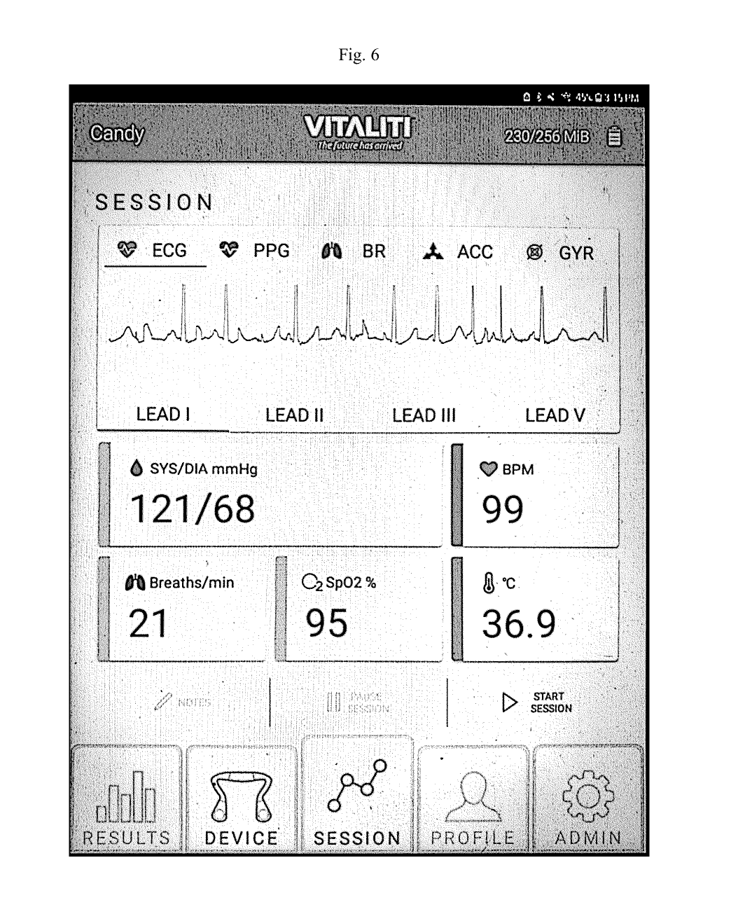

An example of a sample output screen to a professional tablet type external computerized device (300), useful for providing information in preparation for a cardiac CT scan, is shown in FIG. 6.

FIG. 2 shows a closer view of an embodiment of the invention's neck mounted monitor harness device (100). As previously discussed, this comprises two “pods” (110, 120) disposed at opposite ends of the harness. Each “pod” comprises at least one ECG electrode, such as an ECG electrode clip (132), and often these ECG electrode clips are further configured to attach to disposable ECG electrode pads (114,124).

As previously discussed, the two pods at opposite ends of the harness are connected by a semi-rigid U-shaped support “harness” (108). More specifically, in this embodiment, each pod (110, 120) is connected to a semi-rigid support arm (104a, 104b), which may comprise silicone or other semi-rigid material, and these two semi-rigid support arms (104a, 104b) are in turn connected to a center case (106) that can comprise a rigid plastic case, with interior space as needed for additional circuitry and other system components. This additional circuitry can, for example, comprise other device components such a harness processor, harness memory, harness communications interfaces, one or more optional harness electrodes (102), on-off or reset switches, motion sensors, accelerometers, microphones, speakers, and optional connectors to accommodate wired connections to optional devices such as pulse oximeters, oscillometric blood pressure sensors, and the like. In some embodiments, such as shown in FIGS. 1-4, one or more of the pods (110, 120) can also have its own built-in computer display screen, (112, 124). Thus, one or more of these displays can be a standard display or touchscreen device, such as a liquid crystal display (LCD), or another type of display. The display(s) are typically connected to the harness processor so that the harness device (100) can often process and output at least some sensor data without the need of an external computerized device. For example, in FIG. 2 and FIG. 3, one display (112) is showing the patient's pulse rate (a very low 45 beats per minute), while the other display (122) is showing the patient's blood pressure (a somewhat high 135/98).

As previously discussed, in some embodiments, disposable snap-on or clip-on ECG electrode pads (114), (124) are attached to ECG electrode clips (132) mounted on the backside of the two pods (110, 120). Note that in some embodiments, one or more optional harness mounted electrodes (102) may also be a part of the ECG circuitry as well.

The embodiment shown in FIG. 1-4 also comprises a pulse oximeter sensor (162), here configured to mount in the user's ear. This particular pulse oximeter sensor (162) has a light source configured to mount in the user's ear canal (164), a photodetector configured to mount behind the patient's earlobe (166), and a connecting bridge (168) configured to help hold the pulse oximeter sensor in the user's ear. Other pulse oximeter sensor designs, such as finger mounted pulse oximeter sensors (for example, the finger-mounted pulse oximeter designs previously taught by applicant's previous U.S. patent application Ser. Nos. 61/767,839 and 14/186,151), alternate ear mounted pulse oximeter configurations such as those taught by applicant in U.S. patent application Ser. No. 15/268,556, and pulse oximeter sensors configured for other body locations may also be used. The harness device (100) may also connect by wired or wireless data connections to alternative types of sensors such as oscillometric blood pressure monitors, and the like.

Thus in some embodiments, the invention may be an improved ambulatory monitoring system for a human user. This user may be a home user without any medical problems, but often may be a user with medical problems. Thus the “user” is often referred to in the alternative as a “patient”. This system will typically comprise a neck mounted monitor harness (100) comprising a harness processor, harness memory, battery, ECG circuitry, and a harness communications interface. This neck mounted monitor harness will typically comprise a rigid or semi-rigid U-shaped support (104a, 106, 104b) (often comprising one or more silicone tubes 104a, 104b that can bend enough by normal hand strength to facilitate mounting and dismounting of the harness from the neck of a human user, but otherwise in the absence of such hand force will maintain their shape during normal wearing by the user) with a center (106) and two opposite ends (110, 120). These opposite ends (110, 120) are frequently referred to in the alternative as “pods”. In some embodiments, these pods may have their own computer display panels (112, 122), connected to the harness processor, so that at least some signals or physiological parameters may be viewed directly without any need of external computerized devices.

Each opposite end or “pod” of this monitor harness (110, 120) will typically further comprise electrodes (132, and optionally also 114, 124) configured to make electrical contact with the user's front chest skin on opposite sides of the user's sternum. This configuration thus provides at least a single lead ECG electrical connection enabling the ECG circuitry to implement an ECG sensor. Additional ECG electrodes or other type electrodes (102) may also be provided by the monitor harness in alternate positions, such as in the portion of the harness (106) that contacts the back of the user's neck, or as extensions of the pods (110, 120), or elsewhere.

The invention's monitor harness will typically further comprise any of a wired (e.g. a wired connector and wire 160) or wireless (e.g. Bluetooth, Wi-Fi) connection to at least one of a pulse oximeter (162) and/or an oscillometric blood pressure monitor. This allows the harness processor to both further control and receive data from additional sensors outside of the harness, such as the previously discussed pulse oximeter sensor and/or an oscillometric blood pressure sensor.

In a preferred embodiment, the harness processor (often modern electronics employ multiple processors and/or multiple processor cores, so to simplify the grammar, the term “harness processor” should be interpreted as reading on “at least one harness processor”) is configured to drive, over a plurality of patient heartbeats, at least the ECG sensor and any of the pulse oximeter sensor (162) and/or oscillometric blood pressure sensor.

Although the monitor harness may often be configured with factory default system control data such as any of operating-analysis parameters, patient instructions, and physiological target data, in some embodiments these factory default settings may be revised or replaced with additional settings. To allow flexibility, the monitor harness may also be configured to use the harness communications interface (e.g. a connector and suitable hardware software drivers to a wired computer interface, or a wireless interface such as a Bluetooth or Wi-Fi transceiver and supporting drivers) to receive various optional or new system control data such as any of operating-analysis parameters, patient instructions, and physiological target data from an external computerized device, such as (200).

The harness processor will typically also be configured to store any of this system control data, such as the operating-analysis parameters, patient instructions, and physiological target data into the harness memory. The harness processor may then, for example, use these operating-analysis parameters to operate the ECG sensor(s) and other connected devices such as the pulse oximeter and/or the oscillometric blood pressure monitor. These can then collect various types of patent data.

In a preferred embodiment, the harness processor is often further configured; either as a factory default, or as a later uploaded setting, to do additional tasks, such as transmit the patient instructions to the user. This can be done by using the harness processor to transmit these patient instructions to an external computerized device (200), and/or display the patient instructions on the optional pod displays (112, 114), and/or audio output by an optional built-in harness audio speaker, or audio output by, for example, an optional earphone that forms part of an ear-mounted pulse oximeter device (162).

The harness processor is often also further configured, either as a factory fault, or as a later uploaded option, to do additional tasks such as to store data (e.g. sensor data) from any of the previously discussed sensors in the harness memory, and/or transmit this sensor data by using the harness communications interface and/or any optional built-in display screens (112, 122), such as the optional pod display screens.

In a preferred embodiment, the harness processor is often further configured to use the previously discussed operating-analysis parameters to analyze data from any of the previously discussed sensors, and in some embodiments determine if the user's physiological parameters meet previously established physiological criteria (for example, previously established criteria for being in a proper state for a cardiac CT scan). The harness processor is often also configured to use the harness communications interface to transmit at least some of this analyzed data to at least one external computerized device, such as a smartphone, tablet computer, hospital information system, and the like.

Thus, for example, for purposes of the previously discussed preparation for a cardiac CT exam, the user's physiological parameters can comprise physiological parameters optimized for cardiac CT scans. Similarly, for other applications, other physiological parameters may be entered.

More specifically, again using the cardiac CT exam example, these physiological parameters can be a pulse rate of 65 beats per minute or less, ECG parameters showing cardiac sinus rhythm, and any of blood pressure or blood oxygen levels above a preset minimum.

FIG. 3 shows an alternate view of the invention's neck mounted monitor harness device.

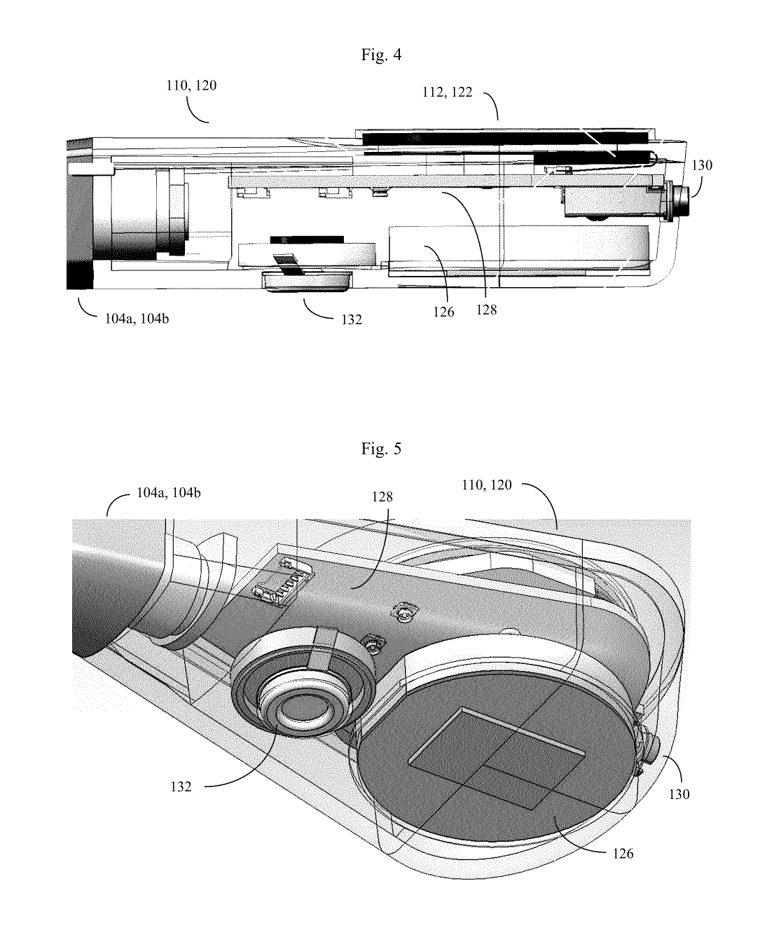

FIG. 4 shows a more detailed 2D cross-sectional drawing of one of the pods (either 110 or 120) of the neck mounted monitor harness (100). Here the display (e.g. LCD display) such as (112 or 122) is shown at the top. The pod's connection to the semi-rigid (or semi-flexible) support arm (104a, or 104b) is also shown. In this embodiment, each pod also has a battery (126) and an internal circuit board (128) with an electrical connector (130) to the outside for charging and/or direct-wired communications purposes. The electrode clip (132) configured to provide an electrical connection to a disposable snap-on ECG electrode pad or dry lead (e.g. 114 or 124) is also shown. The disposable snap-on ECG electrode pad or dry lead is not shown.

In some embodiments, the neck mounted monitor harness (100) is further configured so that the center of the monitor harness (106, 104a, 104b) both mounts and balances across a back of the user's neck, and the opposite ends (e.g. pods 110, 120 plus 104a, 104b) of the monitor harness extend onto opposite sides of the user's chest. Here each opposite end of the monitor harness (e.g. 110 plus 104a, 120 plus 104b) is configured to extend down the user's chest to approximately straddle the user's chest skin proximate to the user's heart. This semi-rigid U-shaped support (108—e.g. 104a, 106, 104b) is typically further configured to self-maintain a bend between about 270 to 360 degrees. This semi-rigid support (108) is typically also configured with sufficient rigidity so that the electrodes (e.g. 114, 124, 132) on the opposite ends of the monitor harness (100) do not rotate, but instead, self-maintain a substantially constant orientation facing the user's skin while the monitoring harness is being worn by the user.

FIG. 5 shows a more detailed 3D drawing showing of one of the pods (either 110 or 120) again showing the ECG electrical connection in more detail. Note that the disposable snap-on ECG sensing pad is not shown. This figure again shows the harness battery (126), which is preferably a rechargeable battery, circuit board (128), and an electrical connector (130) that can be used for any of charging the battery, and/or for use as a wired communications interface to an external computerized device.

For some applications (e.g. to helping prepare a patient for a cardiac CT scan, and other applications), it may be useful to have the system automatically prompt the user to perform certain activities, and to automatically track the physiological results of these activities. For example, consider breath holding results. The system can prompt the user to hold their breath, and the system can then automatically record physiological changes (e.g. change in heartbeat rate, blood oxygen levels, ECG readings) that occur as a result of these user actions.

To implement this, the harness processor and neck mounted monitor harness can be further configured to transmit various visual and/or audio patient instructions to the patient/user (10) to alter their user activity in response to the instructions. (e.g. an audio signal can say: “now try to hold your breath while I count up to 15”) The system's harness processor can also be further configured to analyze the various sensor data to determine if the user's physiological status was detectably altered in response to these patient instructions. Here, it may be useful to further incorporate a microphone or motion sensors into the neck mounted monitor to further automatically determine if the patient has complied with the instructions. For example, an accelerometer or motion sensor can be used, in conjunction with suitable software, to detect if the user's chest is moving during a “hold breath” command.

Returning to the optional pulse oximeter (162). In some embodiments, as previously discussed, the pulse oximeter (162) can further comprise an ear wearable mounted pulse oximeter (162) that is in any of wired or wireless connection with the neck mounted monitor harness (100). This pulse oximeter can be further configured with pulse oximeter light sources, such as various light emitting diodes (LED) configured to emit light (either visible or infrared light) over a plurality of wavelengths. The pulse oximeter (162) will often be further configured so that this light passes through at least some patient tissue, here ear tissue, so that this light passes through at least some patient blood cells and hemoglobin. The pulse oximeter will be further configured with at least one photodetector, configured to receive photodetector signals emitted by these oximeter light sources, usually configured so that the impact of the patient's oxygenated or deoxygenated hemoglobin on this emitted light can be measured, and blood oxygen levels thus determined.

As previously discussed, although the ear wearable mounted pulse oximeter (162) need not have a built-in audio output, in some embodiments it is convenient, particularly for purposes of receiving audio patient instructions, if the ear wearable mounted pulse oximeter (162) further comprises any of an audio output device or microphone configured to be in any of wired or wireless communication with the harness processor. In some embodiments, it is also useful to use the neck mounted device (100) to drive or control an oscillometric blood pressure monitor. This control can be via a wired or wireless link between the neck mounted monitor processor and an external oscillometric blood pressure monitor. Here, for example, the neck mounted monitor processor may be further configured to drive an air pump and valve, which in turn drive a blood pressure monitoring cuff comprising tubing. This air pump and valve can be configured to be either internal to the harness, or external to the harness. In FIG. 1-5, the air pump, and valve, should be assumed to be external to the harness. Indeed, the harness processor can drive the air pump and valve via a wireless connection, such as a Bluetooth or Wi-Fi connection, as desired. As another alternative, a wired data line may also be used. In either case, the neck mounted monitor processor may be further configured to receive input from this at least one oscillometric blood pressure detector, and use this input to monitor pulse input from the blood pressure monitoring cuff.

As will be discussed in more detail shortly, in some embodiments, it is useful to further configure the harness processor to use the harness communications interface (such as a wired or wireless interface) to transmit a message to an outside computerized device (200 or other device) when the analyzed data meet the previously established physiological criteria.

FIG. 6 shows an example touchscreen from an external handheld computerized device (here a tablet computer 300), used to display patent data obtained from various sensors on or controlled by the neck mounted monitor harness (100), and in this example wirelessly transmitted from the neck mounted monitor harness (100) to the external handheld computerized device (200 or 300).

Here the neck mounted monitor harness (100) is transmitting ECG data, blood pressure data (from an optional oscillometric blood pressure monitor), heart rate in beats per minute, blood oxygen levels (O₂ SpO₂%) from an optional ear mounted pulse oximeter, and other information such as the number of breaths per minute from an optional harness mounted motion sensor or microphone sensor, and patient temperature from an optional temperature sensor, here mounted in the ear-mounted pulse oximeter (162).

More specifically, in some embodiments, such as for the previously discussed purpose of preparing patients for cardiac CT scans, the system can further comprise a professional external computerized device (300, also see FIG. 6) in direct or indirect wireless communication with the harness processor. This professional external computerized device (300) can itself be a tablet computer such as an iOS or Android tablet computer, an augmented reality headset, or another type of computerized device, and will typically comprise a professional graphical user interface (see FIG. 6). This professional external computerized device (300) can further be configured to perform additional functions. These can include using the professional graphical user interface to transmit various data, such as the previously discussed operating-analysis parameters, patient instructions, and physiological target data to the harness processor. Alternatively or additionally, the professional user interface can also be used to receive either raw data or harness processor data from the harness processor and to display some or all of this raw data and/or analyzed data on the professional graphical user interface.

Other types of external computerized devices can also be a part of the system. For example, in some embodiments, the system can further comprise a patient-user external computerized device for use by the patient as well (see FIG. 1, 200). This patient-user computerized device may also be an iOS or Android type tablet computer or smartphone, or another type of computerized device, and may be in either direct or indirect (e.g. by intermediate routers, servers, and the like) wireless communication either the harness processor, and the professional external computerized device (300). This user external computerized device (200) will typically also comprise a user graphical user interface. The user external computerized device (200) can be configured to use the user graphical user interface to query an ambulatory patient (10) or caregiver with various queries regarding her health history and various medical conditions. The user external computerized device (200) can then be used to transmit the patient's (or caregiver's) answers to these questions to any combination of the harness processor (100), the professional external computerized device (300), or one or more other external computerized devices. Alternatively, a professional computerized device (300) may be used to perform the above functions.

Specific applications for preparing a patient for a cardiac CT scan:

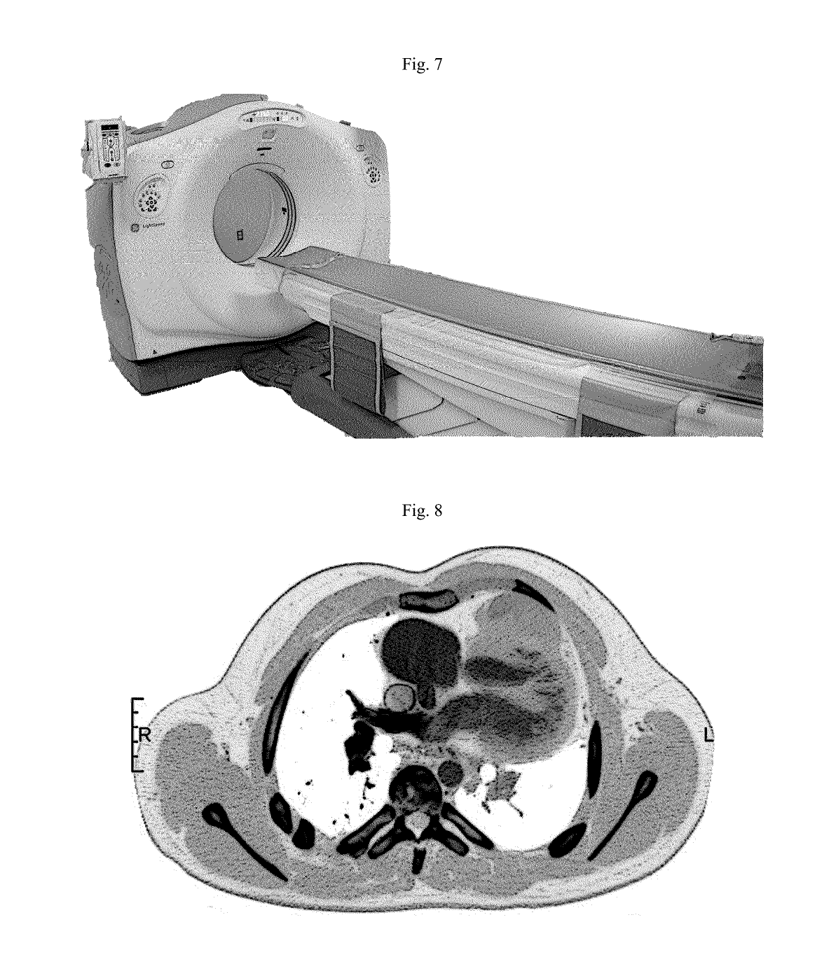

FIG. 7 shows an example of a CT scanner, here a GE Lightspeed CT scanner. This image has been modified from a source image from Wikipedia.org, made available courtesy of copyright holder daveyin, and is here provided under their Creative Commons Attribution 2.0 Generic license.

FIG. 8 shows an inverted grayscale example of a cardiac CT scan. This image has been modified from a source image of an “Axial CT image in a patient with congenital tricuspid atresia post bidirectional Glenn shunt (SVC to right pulmonary artery and extracardiac Fontan conduit (IVC to pulmonary artery) with anomalous origin of the left coronary artery from the non coronary cusp and fistula to the right ventricle”, uploaded on Wikimedia.org by copyright holder Jto410, and made available under their Creative Commons Attribution-Share Alike 3.0 Unported license.

The goal is to produce a very sharp image, useful for medical diagnostic purposes. To do this, patient movement should be avoided during the scan, to the greatest extent possible consistent with patient comfort and safety.

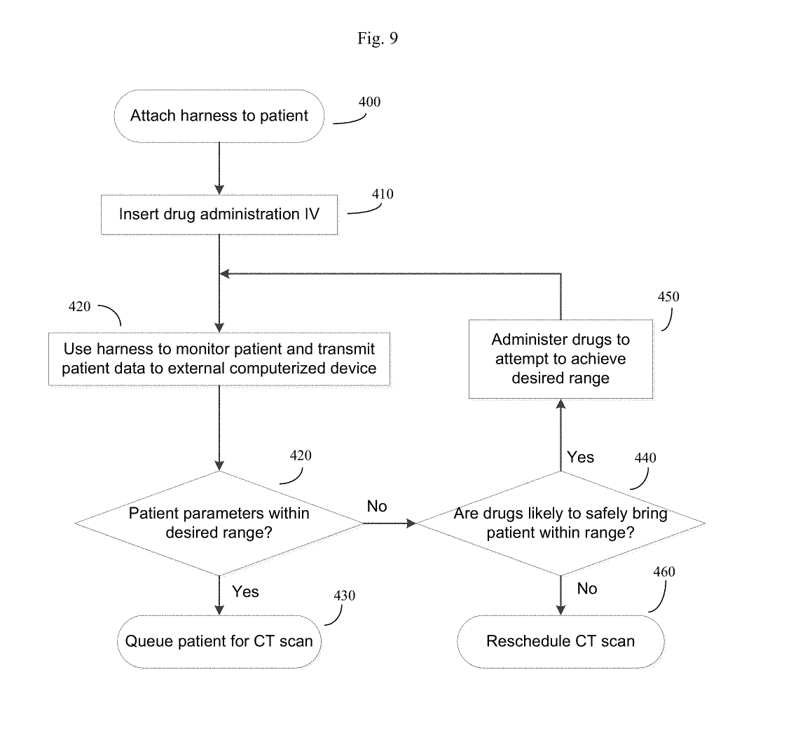

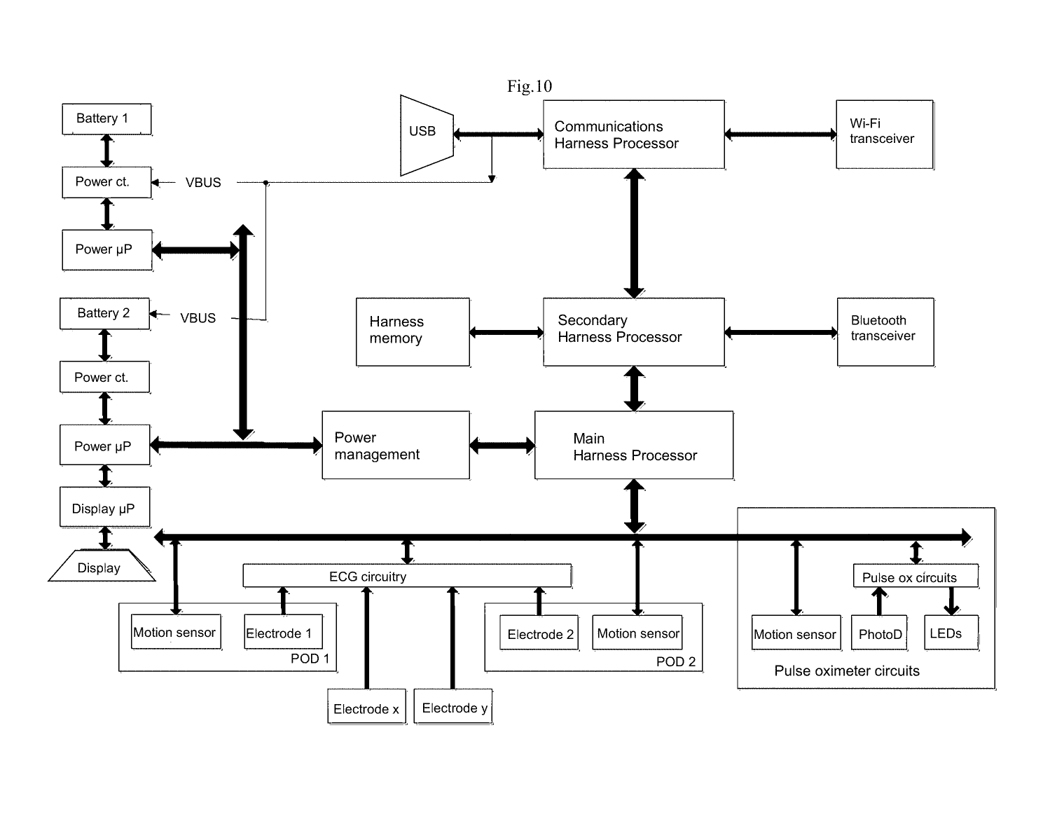

FIG. 9 shows a simplified flow chart showing how the invention's neck mounted monitor harness device (100) can be used to help prepare patients for cardiac CT scans.

In this example, in upon arrival in the imaging department (400), a patient (10) can be registered by clerical staff, then brought into a CT scan area, given a gown, and equipped with the invention's neck mounted monitoring harness device (100). As desired, additional demographic information, pre-scan screening questionnaires, and other information can be entered onto a device such as an external computerized device tablet computer or another type of device (e.g. 200 or 300).

In some protocols (410), an IV (intravenous therapy line) can also be inserted into the patient (10) to facilitate drug administration to bring the patients' heartbeat rate and other physiological parameters into the range desired for cardiac CT scanning. Various other nursing administration and physician oversight functions can also be done around this time, such as entering or verifying the patient's demographic information, pre-scan screening information, and other relevant information into suitable computerized devices (300). Additionally, upon review of this information, physicians may provide instructions and orders as to the desired patient parameter ranges, and medications that may be used to achieve these desired ranges. For example, a physician may specify that the desired range is a heart rate while the patient holds their breath of under 60 beats per minute, and specify that certain doses of certain medications such as metoprolol (a beta-blocker) or nitroglycerine may be used to put the patient parameters into the desired range.

Indeed, in more advanced versions of the invention, some embodiments of the harness mounted device may additionally be configured to control the IV administration of medications to meet the desired CT preparatory objectives, or even administer appropriate voltages through the ECG electrodes in order to convert the patient's heart rate to a regular ECG sinus rhythm. In some use cases, the control of a broad range of IV administered drugs can also be controlled by various parameters detected by the invention's harness device, including timed infusions.

To help with functions such as breath holding tests, in some embodiments, the harness processor and neck mounted monitor harness (100) can be used to transmit various visual or audio patient instructions (e.g. by way of displays 112, 124, computerized device 200, or ear mounted pulse oximeter audio output (162) to the patient-user (10). These instructions can, for example, tell the user to alter her activity in response to the patient instructions (e.g. “now hold your breath for 15 seconds”). In some embodiments, the harness processor can be further used to analyze some or all of the previously discussed sensor data to determine if the user (patient 10) had an altered physiological status in response to these instructions.

The invention's neck mounted monitoring harness device (100) may be used to determine (or at least confirm) if the patient is initially presenting with physiological parameters (e.g. desired pulse) in the desired range (420). Typically the patient's physiological parameters will initially not be optimal for cardiac CT scanning. As part of the process of optimizing the patient's physiological parameters for cardiac CT scanning, the harness monitor (100) will be used to monitor the patient continuously (or nearly continuously) for periods of time often ranging between 30 minutes to several hours. During this time, in addition obtaining normal (not holding the breath) physiological data, the patient, can also be manually or automatically (e.g. by using prompts provided by device 100, or other method) instructed to periodically (e.g. every 15 minutes) hold their breath so as to also produce breath holding physiological data. In some embodiments, either the harness monitor device itself (100) or an external computerized device (200, 300) receiving data from the harness monitor device, can also automatically notify healthcare personnel if the patient parameters are now in the desired range (420). If these parameters are acceptable, then the patient can then be manually or automatically scheduled for the CT scan (430).

However if the patient physiological data (parameters) are not in the desired range (420), and if the physician has determined that drugs are likely to bring the patient within range (440), and the appropriate orders have been entered into the system, then an external computerized device, usually a hospital or clinic computer system (300) that may also be in communication with the harness medical device 100), can be configured to authorize (450) appropriate drugs to be administered to the patient. The process can then repeat, often for time durations between 1-6 hours. However, if according to physician's judgment or standing orders entered into the system (440), the allowable time to achieve a desired patient physiological parameter range has been exceeded, or the allowable drug dosage has been exceeded, or other criteria are not favorable, then the patient may be automatically or manually excused and the CT scan likely rescheduled to a later date (460). This scenario can also occur if the harness monitor device (100) also provides data suggesting that the patient needs to be more quickly discharged from the protocol (e.g. the harness monitor device (100) shows that the patient's heart rate is too fast, blood oxygen levels, are too low, ECG not showing proper sinus rhythm), or evidence of other problems is found.

FIG. 10 shows an electrical circuit diagram of one embodiment of the invention's neck mounted monitor harness device (100). In particular, FIG. 10 shows more details of the invention's harness processor(s), harness memory, ECG circuitry, and harness communications interface (here configured to drive both wired USB communications interfaces, and wireless Wi-Fi communications interfaces. In this embodiment, the neck mounted monitor harness device (100) is also configured with circuitry to drive an ear-mounted pulse oximeter. In this embodiment, the neck mounted monitor harness device is also configured with additional sensors, such as temperature sensors, as well as various motion sensors such as accelerometer and gyroscopic type motion sensors. Certain aspects of the system's battery and power management circuitry, and the display circuitry, are also shown. In alternative embodiments, other circuitry, such as haptic vibration output, and audible (audio) alarms is also provided.

1. An ambulatory monitoring system for a human user, comprising:
a neck mounted monitor harness comprising a harness processor, harness memory, battery, ECG circuitry, and a harness communications interface, said neck mounted monitor harness further comprising a semi-rigid U-shaped support with a center and two opposite ends;
each opposite end of said monitor harness further comprising electrodes configured to make electrical contact with said user's front chest skin on opposite sides of said user's sternum, thus providing at least a single lead ECG electrical connection enabling said ECG circuitry to implement an ECG sensor;
said monitor harness further comprising any of a wired or wireless connection to at least one of a pulse oximeter and an oscillometric blood pressure monitor so said harness processor may further control and receive data from any of a pulse oximeter sensor and an oscillometric blood pressure sensor;
said harness processor configured to drive, over a plurality of patient heart beats, said ECG sensor and any of said pulse oximeter sensor and said oscillometric blood pressure sensor;
said monitor harness further configured to use said harness communications interface to receive any of operating-analysis parameters, patient instructions, and physiological target data from an external computerized device, store any of said operating-analysis parameters, patient instructions, and physiological target data in harness memory, and use said operating-analysis parameters to operate said ECG sensor and any of said pulse oximeter and oscillometric blood pressure monitor, and said harness processor further perform any of:
transmit said patient instructions to said user;
store data from any of said sensors in harness memory and transmit said data using said harness communications interface;
use said operating-analysis parameters to analyze data from any of said sensors to determine if said user's physiological parameters meet previously established physiological criteria; and using said harness communications interface to transmit said analyzed data to at least one external computerized device.

2. The system of claim 1, wherein said user's physiological parameters comprise physiological parameters optimized for cardiac CT scans.

3. The system of claim 2, wherein said physiological parameters comprise a pulse rate of 65 beats per minute or less, often determined by the speed at which the CT scanner obtains its image, among other factors, ECG parameters showing cardiac sinus rhythm, and any of blood pressure or blood oxygen levels above a preset minimum.

4. The system of claim 1, wherein said harness processor is further configured to use said harness communications interface to transmit a message to an outside computerized device when said analyzed data meet said previously established physiological criteria.

5. The system of claim 1, wherein said harness processor and neck mounted monitor harness is further configured to transmit any of visual or audio patient instructions to said user to alter a user activity in response to said instructions, and wherein said harness processor is further configured to analyze any of said sensor data to determine altered user physiological status in response to said patient instructions.

6. The system of claim 1, wherein said pulse oximeter further comprises an ear wearable mounted pulse oximeter that is in any of wired or wireless connection with said neck mounted monitor harness, and wherein said pulse oximeter is further configured with pulse oximeter light sources that emit light over a plurality of wavelengths, and to receive photodetector signals over emitted by said oximeter light sources.

7. The system of claim 6, wherein said ear wearable mounted pulse oximeter further comprises any of an audio output device or microphone configured to be in any of wired or wireless communication with said harness processor.

8. The system of claim 1, wherein said neck mounted monitor processor is further configured to drive an air pump and valve for driving a blood pressure monitoring cuff comprising tubing, said air pump, and valve being configured either internal or external to said harness;
said neck mounted monitor processor further configured to receive input from at least one oscillometric blood pressure detector to monitor pulse input from said blood pressure monitoring cuff.

9. The system of claim 1, wherein said system further comprises a professional external computerized device in direct or indirect wireless communication with said harness processor, said professional external computerized device comprising a professional graphical user interface, said professional external computerized device configured to perform at least one of:
a) use said professional graphical user interface to transmit any of operating-analysis parameters, patient instructions, and physiological target data to said harness processor; and
b) receive any of said data and analyzed data from said harness processor, and to display any of said data and analyzed data on said professional graphical user interface.

10. The system of claim 9, wherein said system further comprises a user external computerized device in direct or indirect wireless communication with any of said harness processor and said professional external computerized device, said user external computerized device comprising a user graphical user interface, said user external computerized device configured to use said user graphical user interface to query an ambulatory patient with a plurality of queries regarding a health history pertaining to a plurality of medical conditions of said ambulatory patient, and transmit answers to said queries to any of said harness processor and said professional external computerized device.

11. The system of claim 1, wherein said neck mounted monitor harness is further configured so that said center of said monitor harness both mounts and balances across a back of said user's neck, and said opposite ends of said monitor harness extend onto opposite sides of said user's chest;
each opposite end of said monitor harness configured to extend down said user's chest to approximately straddle chest skin proximate said user's heart; and
said semi-rigid U-shaped support further configured to self-maintain a bend between 270 and 360 degrees, and further configured so that said electrodes on said opposite ends of said monitor harness do not rotate, but instead self-maintain a substantially constant orientation facing said user's skin while said monitoring harness is being worn by said user.

12. The system of claim 1, wherein said harness communications interface is any of wired computer interface or a wireless transceiver.

13. An ambulatory monitoring system for a human user, comprising:
a neck mounted monitor harness comprising a harness processor, harness memory, battery, ECG circuitry, and a harness communications interface, said neck mounted monitor harness further comprising a semi-rigid U-shaped support with a center and two opposite ends;
said neck mounted monitor harness is further configured so that said center of said monitor harness both mounts and balances across a back of said user's neck, and said opposite ends of said monitor harness extend onto opposite sides of said user's chest;
each opposite end of said monitor harness configured to extend down said user's chest to approximately straddle chest skin proximate said user's heart;
each opposite end of said monitor harness further comprising electrodes configured to make electrical contact with said user's front chest skin on opposite sides of said user's sternum, thus providing at least a single lead ECG electrical connection enabling said ECG circuitry to implement an ECG sensor;
said monitor harness further comprising any of a wired or wireless connection to at least one of a pulse oximeter and an oscillometric blood pressure monitor so said harness processor may further control and receive data from any of a pulse oximeter sensor and an oscillometric blood pressure sensor;
said semi-rigid U-shaped support further configured to self-maintain a bend between 270 and 360 degrees, and further configured so that said electrodes on said opposite ends of said monitor harness do not rotate, but instead self-maintain a substantially constant orientation facing said user's skin while said monitoring harness is being worn by said user;
said neck mounted monitor processor further configured to drive an air pump and valve for driving a blood pressure monitoring cuff comprising tubing, said air pump, and valve being configured either internal or external to said harness;
said neck mounted monitor processor further configured to receive input from at least one oscillometric blood pressure detector to monitor pulse input from said blood pressure monitoring cuff;
said harness processor configured to drive, over a plurality of patient heart beats, said ECG sensor and any of said pulse oximeter sensor and said oscillometric blood pressure sensor;
said monitor harness further configured to use said harness communications interface to receive any of operating-analysis parameters, patient instructions, and physiological target data from an external computerized device, store any of said operating-analysis parameters, patient instructions, and physiological target data in harness memory, and use said operating-analysis parameters to operate said ECG sensor and any of said pulse oximeter and oscillometric blood pressure monitor, and said harness processor further perform any of:
transmit said patient instructions to said user;
store data from any of said sensors in harness memory and transmit said data using said harness communications interface;
use said operating-analysis parameters to analyze data from any of said sensors to determine if said user's physiological parameters meet previously established physiological criteria; and using said harness communications interface to transmit said analyzed data to at least one external computerized device;
wherein said system further comprises a professional external computerized device in direct or indirect wireless communication with said harness processor, said professional external computerized device comprising a professional graphical user interface, said professional external computerized device configured to perform at least one of:
a) use said professional graphical user interface to transmit any of operating-analysis parameters, patient instructions, and physiological target data to said harness processor; and
b) receive any of said data and analyzed data from said harness processor, and to display any of said data and analyzed data on said professional graphical user interface.

14. The system of claim 13, wherein said user's physiological parameters comprise physiological parameters optimized for cardiac CT scans.

15. The system of claim 13, wherein said harness processor and neck mounted monitor harness is further configured to transmit any of visual or audio patient instructions to said user to alter a user activity in response to said instructions, and wherein said harness processor is further configured to analyze any of said sensor data to determine altered user physiological status in response to said patient instructions.

16. The system of claim 13, wherein said pulse oximeter further comprises an ear wearable mounted pulse oximeter that is in any of wired or wireless connection with said neck mounted monitor harness, and wherein said pulse oximeter is further configured with pulse oximeter light sources that emit light over a plurality of wavelengths, and to receive photodetector signals over emitted by said oximeter light sources.

17. A method of monitoring an ambulatory human user for at least one physiological criteria, said method comprising:
fitting said human user with a neck mounted monitor harness comprising a harness processor, harness memory, battery, ECG circuitry, and a harness communications interface, said neck mounted monitor harness further comprising a semi-rigid U-shaped support with a center and two opposite ends;
said neck mounted monitor harness is further configured so that said center of said monitor harness both mounts and balances across a back of said user's neck, and said opposite ends of said monitor harness extend onto opposite sides of said user's chest;
each opposite end of said monitor harness configured to extend down said user's chest to approximately straddle chest skin proximate said user's heart;
each opposite end of said monitor harness further comprising electrodes configured to make electrical contact with said user's front chest skin on opposite sides of said user's sternum, thus providing at least a single lead ECG electrical connection enabling said ECG circuitry to implement an ECG sensor;
said monitor harness further comprising any of a wired or wireless connection to at least one of a pulse oximeter and an oscillometric blood pressure monitor so said harness processor may further control and receive data from any of a pulse oximeter sensor and an oscillometric blood pressure sensor;
said semi-rigid U-shaped support further configured to self-maintain a bend between 270 and 360 degrees, and further configured so that said electrodes on said opposite ends of said monitor harness do not rotate, but instead self-maintain a substantially constant orientation facing said user's skin while said monitoring harness is being worn by said user;
using said harness processor to drive, over a plurality of patient heart beats, said ECG sensor and any of said pulse oximeter sensor and said oscillometric blood pressure sensor;
using said monitor harness and said harness communications interface to receive any of operating-analysis parameters, patient instructions, and physiological target data from an external computerized device, store any of said operating-analysis parameters, patient instructions, and physiological target data in harness memory, and use said operating-analysis parameters to operate said ECG sensor and any of said pulse oximeter and oscillometric blood pressure monitor, and further using said harness processor further perform any of:
transmitting said patient instructions to said user;
storing data from any of said sensors in harness memory and transmitting said data using said harness communications interface;
using said operating-analysis parameters to analyze data from any of said sensors to determine if said user's physiological parameters meet previously established physiological criteria; and transmitting said analyzed data using said harness communications interface to at least one external computerized device.

18. The method of claim 17, wherein said user's physiological parameters comprise physiological parameters optimized for cardiac CT scans.

19. The method of claim 17, further using said neck mounted monitor processor to drive an air pump and valve for driving a blood pressure monitoring cuff comprising tubing, said air pump, and valve being configured either internal or external to said harness;
further using said neck mounted monitor processor further to receive input from at least one oscillometric blood pressure detector, and using said input from at least one oscillometric blood pressure detector to monitor pulse input from said blood pressure monitoring cuff.

20. The method of claim 17, further using said harness processor and neck mounted monitor harness to transmit any of visual or audio patient instructions to said user to alter a user activity in response to said patient instructions, and further using said harness processor to analyze any of said sensor data to determine altered user physiological status in response to said patient instructions.

21. The method of claim 17, further using a professional external computerized device in direct or indirect wireless communication with said harness processor, said professional external computerized device comprising a professional graphical user interface, to perform at least one of:
a) using said professional graphical user interface to transmit any of operating-analysis parameters, patient instructions, and physiological target data to said harness processor; and
b) receiving any of said data and analyzed data from said harness processor, and displaying display any of said data and analyzed data on said professional graphical user interface.

22. The system of claim 17, wherein said harness communications interface is any of wired computer interface or a wireless transceiver.
